(19) United States
(12) Patent Application Publication
(10) Pub. No.: US 2015/0182131 A1
(43) Pub. Date: Jul. 2, 2015

(54) NEONATAL HEALTH CARE MONITORING SYSTEM

(71) Applicants: Mohamed R. Mahfouz, Knoxville, TN (US); Gary To, Knoxville, TN (US); Mark S. Gaylord, Knoxville, TN (US); Vichien Lorch, Knoxville, TN (US)

(72) Inventors: Mohamed R. Mahfouz, Knoxville, TN (US); Gary To, Knoxville, TN (US); Mark S. Gaylord, Knoxville, TN (US); Vichien Lorch, Knoxville, TN (US)

(21) Appl. No.: 14/526,003

(22) Filed: Oct. 28, 2014

Related U.S. Application Data
(60) Provisional application No. 61/896,444, filed on Oct. 28, 2013.

(51) Int. Cl. A61B 5/0205; A61H 23/00; A61B 5/00; A61B 7/00; A61B 7/04

(52) U.S. Cl. CPC A61B 5/0205; A61B 7/003; A61B 7/008; A61B 7/04; A61B 5/42; A61B 5/6804; A61B 5/6892; A61B 5/7282; A61B 5/742; A61B 5/746; A61H 23/00; A61B 5/0826

(57) ABSTRACT

A neonatal monitoring system comprising: (a) a substrate comprising at least one of bedding and a garment for a patient, the substrate including at least four vibration sensors and a pressure sensor array; (b) a computer communicatively coupled to the at least four vibration sensors to receive output data from each of the at least four vibration sensors, where the computer includes at least one algorithm for filtering and conditioning output data received from the at least four vibration sensors; and, (c) a visual, display communicatively coupled to the computer for displaying information regarding the patient condition.

### CROSS REFERENCE TO RELATED APPLICATIONS

The present application claims the benefit of U.S. Provisional Patent Application Ser. No. 11/896,444, entitled, “NEONATAL HEALTHCARE MONITORING SYSTEM,” filed Oct. 28, 2013, the disclosure of which is incorporated herein by reference.

### RELATED ART

1. Field of the Invention

The present invention is directed to a neonatal health care monitoring system.

2. Introduction to the Invention

Infection is one of the leading causes of death in the neonatal intensive care unit. Current monitoring systems use sensors with a medical adhesive that causes skin trauma, which itself introduces a pathway for infection. There are several high risks factors contributing to the increase in the neonatal intensive care unit (NICU) infection rate, which include skin breakdown from medical adhesive, contamination of a stethoscope, and the warm and humid environment within the incubator that facilitates bacteria and fungi growth. Infection is commonly caused by skin trauma or contamination of the microenvironment of the incubator. A study reported that 65% of extremely low birth weight (ELBW) neonate survivors (<1000 g, approximately 24-30 weeks gestation) had developed at least one infection during their hospitalization.

One of the most common symptoms observed in preterm neonate is apnea of prematurity (AOP), namely, the cessation of breathing for periods of 20 seconds or greater. The effects of a cessation in respiration are detrimental to the health of the infant, stemming from conditions such as hypoxemia and bradycardia, which often accompany apneic episodes. Prolonged apnea and bradycardia, can decrease the systemic blood, pressure and lead to cerebral hypo-perfusion, which may contribute to hypoxic ischemic injury to the developing brain or other organs.

Current monitoring systems observe electrocardiography, respiratory rate, oxygen saturation (Sp0₂), and noninvasive blood pressure (NBP), the outputs of which are depicted on a visual display (e.g., a General Electric Dash monitor). When apnea is detected by using these conditions, the infant has ceased breathing for at least 20 seconds, at which point the alert system sounds an alarm, thereby alerting the clinical staff of an apneic episode. The clinician provides physical stimulation to the neonate. But this stimulation requires disrupting the microenvironment within the incubator, which increases the chance for contamination.

Medical adhesives have been used extensively to secure medical equipment onto patients. However, due to the under-developed stratum corneum of ELBW neonates, a single adhesive removal will disrupt and compromise the skin barrier function of the premature neonate. This single adhesive removal causes skin trauma and significantly increases the risk of bacterial and fungal infection. One of the most frequently used vitals monitoring device in the NICU is the electrocardiogram (ECG), where medical adhesives such as plastic tapes, pectin barriers, or hydrogel adhesives are used to secure electrodes on patients. A neonatal skin care study found that the first two methods induced significant risk of skin disruption based on trans-epidermal water loss (TEWL) and colorimeteric measurements. In this same study, although commercial available hydrogel adhesives do not cause trauma, they are unsuitable for long-term critical monitoring as 24% of the gel detached after the first 24 hours. Adhesive removal solvents have also been shown to cause epidermal injury.

Another risk factor for neonate infection is the contamination of stethoscopes. It has been shown from multiple studies that on average over 80% of stethoscopes were contaminated with bacteria. Acoustic assessment of the heart, lungs, and bowel with stethoscopes is crucial in diagnosing many symptoms or conditions of neonates. Heart murmur, hyperactive, hypoactive, or missing bowel sounds can be an indicator for many disease. Special designed stethoscopes with lengthen tubes are required to reach the neonate inside the incubator. The neonate is checked by the clinician multiple times during the day, where each examination disrupts the microenvironment in the incubator.

ELBW neonates often require intubation at birth. Malposition and partial obstruction of the endotracheal tube (ETT), which is diagnosed with stethoscopes, is commonly observed and can be life threating. However, it is impractical to assign a caregiver to every patient in the NICU to continuously listen for the heart, lung, and bowel sounds. In addition, it is currently impossible to quantitatively measure lung volume continuously without the use of an invasive ventilator.

The first component of the NICU healthcare system is to replace the infant's current nasal cannula with a nearly analogous nasal cannula that has the added ability to monitor, utilizing a side stream sampling method of exhaled CO₂. This method of patient monitoring is known as capnography. This sensing component is used to monitor for instances of apnea, which is connected to a processing unit within the incubator. Computer aided diagnostics are performed based on, but not limited to, the input from the capnograph. If an apnea condition is diagnosed, a wireless signal is sent to a stimulation device. For example, a vibrator inside the neonate's garment to simulate the physical stimulation from a clinician.

Vibroarthrography is a non-invasive diagnostic technique that monitors the in-vivo vibration of the human body, which was initially employed in detecting the vibration within human joints during motion. A highly sensitive, high dynamic range vibration sensor can be used to monitor the mechanical movement of the heart valves, the expanding and contracting motion of the lungs, as well as the vibration from the bowel's motion. A system incorporating a highly sensitive, high dynamic range vibration sensor allows the caregiver to select the frequency of monitoring to aid in diagnosis of the interested organ. For example, to identify a heart murmur, the caregiver can restrict the audio output to a low frequency range so that the sound of the heart tones will not be included at the output.

A vibroarthrography system can substitute the use of ECG and stethoscopes on fragile neonates. This system processes the sensor data and provides audio feedback in real time or time delayed for future analysis. The microenvironment is maintained without opening the incubator while these measurements are made. Moreover, a significant advantage of the system is that it provides a solution for non-invasive monitoring on physiological measurements. For example, the sensors on the lungs are used to determine the tidal volume and residual capacity once the initial readings from the sensors are calibrated to the parameters obtained from the ventilator.

One exemplary design consists of four or more vibration sensing elements. The vibration sensing elements are placed in proximity to the heart, the left and right sides of the lungs, and the bowel of the patient. The vibration sensors measure internal vibrations of the patient caused from heartbeat, breathing, and bowel movement. In sum, this exemplary system operates as multiple stethoscopes for autonomous and continuous monitoring.

Although there are non-invasive, non-intrusive methods to obtain an infant's body temperature, the most common method utilizes a sensor placed at a single location on the infant. But this single sensor may not be able to detect complications such as peripheral vascular diseases. Infrared thermal cameras are particularly useful in monitoring both body temperature and movement of a patient. In the case of an infant, a thermal image map can help the clinician diagnosis certain vascular diseases.

The computer aided diagnostic system is the centralized data processing unit. The outputs from various sensors are connected to this system. The system automatically tracks and monitors conditions of one or more patients. Based on inputs to the sensing system, a classification software suite using a multi-dimensional classification algorithm is used to detect and notify a caretaker if an anomaly is detected.

The last component is the feedback and alert system. The feedback system is aimed to provide simple feedback to the patient without interfering with the incubator's environment. For example, if apnea is detected, a physical stimulation device, such as a vibration motor embedded within clothing or bedding of the patient, is directed to provide physical stimulation to restore breathing.

### BRIEF DESCRIPTION OF THE DRAWINGS

FIG. 1 is a schematic diagram of an exemplary patient monitoring system in accordance with the instant disclosure.

FIG. 2 is a top view of a first exemplary embodiment for embedding vibration sensors within a patient garment, along with other top views showing the garment wrapped around an extremely low birthrate patient.

FIG. 3 is a top view of a second exemplary embodiment for embedding vibration sensors within a patient garment, along with a top view showing the garment wrapped around a very low birthrate patient.

FIG. 4 is the data collected by the vibration sensors from healthy adult placed closed to the heart, with the blue line indicating raw data, the red line indicating filtered output of the raw data, and the remaining data is the vibration signature of the closing the heart valves of the healthy adult.

FIG. 5 is a zoomed-in version of the data signals in FIG. 4.

FIG. 6 is the data collected by the vibration sensors from healthy adult placed close to the right lung, with the blue line indicating raw data and the red line indicating the filtered output of the raw data signal.

FIG. 7 is a system flow diagram for the apnea monitoring and alerting system as part of the exemplary patient monitoring system of FIG. 1.

FIG. 8 is a schematic diagram showing the diagnostic algorithms use of signal classification to diagnose various heart conditions.

### DETAILED DESCRIPTION

The exemplary embodiments of the present disclosure are described and illustrated below to encompass a neonatal health care monitoring system. Of course, it will be apparent to those of ordinary skill in the art that the embodiments discussed below are exemplary in nature and may be reconfigured without departing from the scope and spirit of the present disclosure. However, for clarity and precision, the exemplary embodiments as discussed below may include optional steps, methods, and features that one of ordinary skill should recognize as not being a requisite to fall within the scope of the present disclosure.

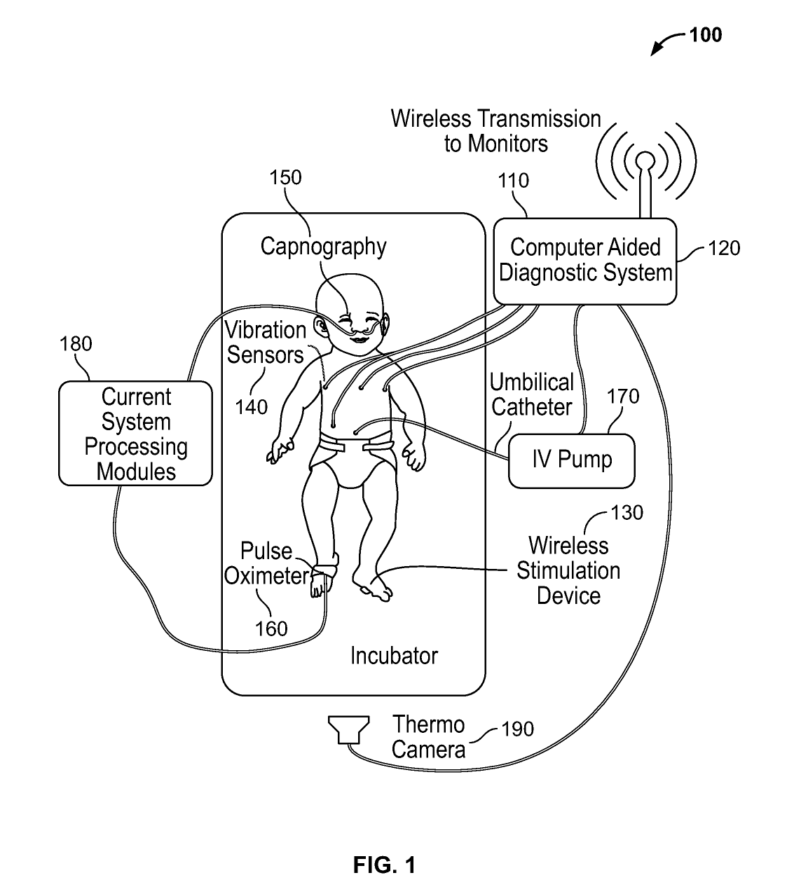

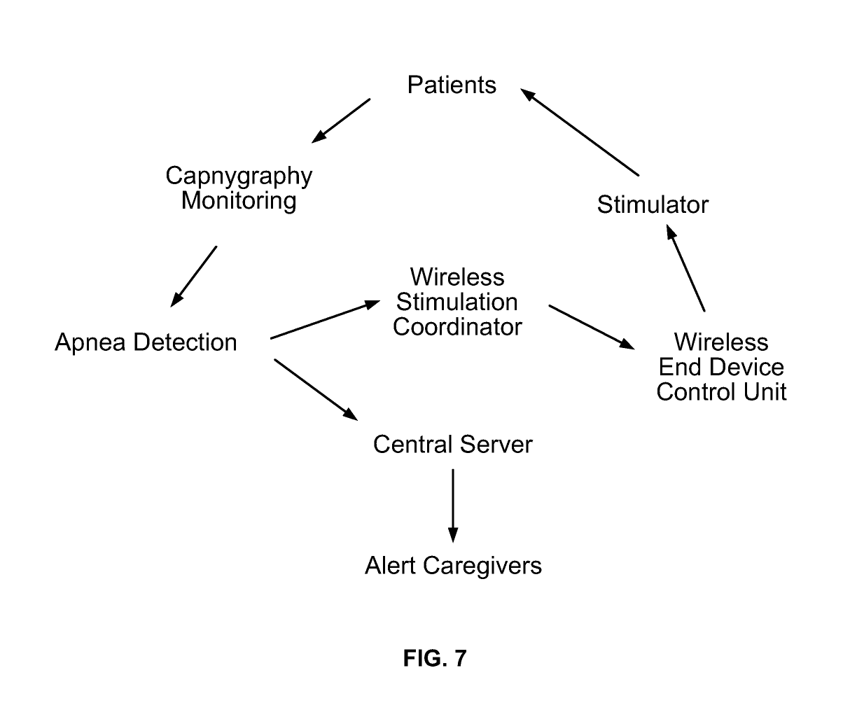

Referencing FIGS. 1 and 7, an exemplary patient monitoring system 100 includes a plurality of vital signs monitors, the outputs from which are connected to a computer aided diagnostic computer (CADC) 110. The patient monitoring system 100 monitors and performs diagnosis autonomously and continuously on the patient, in exemplary form a premature baby in a neonatal intensive care unit (NICU). In addition to the CADC 110, the monitoring system 100 also includes an alert and feedback component 120 (which may be part of the CADC 110), which consists of a plurality of actuators 130 that are triggered by corresponding detected symptoms and is operative to alert a caregiver, such as a neonatal nurse, based upon the detected symptoms. In addition, drug administration and physical stimulation for abnormal vitals, such as apnea, may be achieved by activating the actuators with or without human intervention. Present day vitals sensing and diagnostic system 180 may also be used as a peripheral input(s) to the CADC 110. Examples of monitoring devices utilized as part of the present day vitals sensing and diagnostic system 180 include, without limitation, breathing monitors 150 (e.g., capnography) oxygen saturation rate monitors 160 (e.g., pulse oximeter), and infrared thermal imaging cameras 190.

a core component of the patient monitoring system 100 comprises a substrate and a flexible component, which are embedded with multiple vibration sensors 140 are used to monitor the internal vibration of the heart, lungs, and bowel. In exemplary form, the vibration sensors 140 may be based on, but are not limited to, piezoelectric materials. By way of example, the vibration sensors 140 are operative to produce a charge, voltage, or current from the vibration detected by each sensor. A readout and signal conditioning unit may be utilized to condition the raw signal for an analog to digital converter as part of the CADC 110. The resulting digital data is then processed by the CADC 110 to generate feedback regarding the condition of the patient. This feedback may be in the form of outputs that are visually displayed on diagnostic monitors to provide real-time updates concerning changes in the patient's condition.

the exemplary patient monitoring system 100 allows for digitalization of traditional qualitative assessments of the patient. The data collected from the vibration sensors 140 is fed to the CADC, where a fully automatic diagnostic program assesses the collected data (in addition to other data captured from existing monitors) and diagnoses the patient's condition at least in part concerning the heart, lung, and bowel movements. The system 100 may be used for extended periods of time to diagnose and generate responsive actions (e.g., increase/decrease intravenous flow via an IV pump 170, activate a vibrator 130, etc. O without intervention into the incubator (except for human intervention, if necessary). In addition, as will be discussed in more detail hereafter, utilization of the vibration sensors 140 does not require utilization of medical adhesives, thereby greatly limiting the chance of skin trauma, contamination, and infections that present day sensors require. In addition, the system 100 may be used as a training device and utilized in environments besides that of an NICU, such as pediatrics.

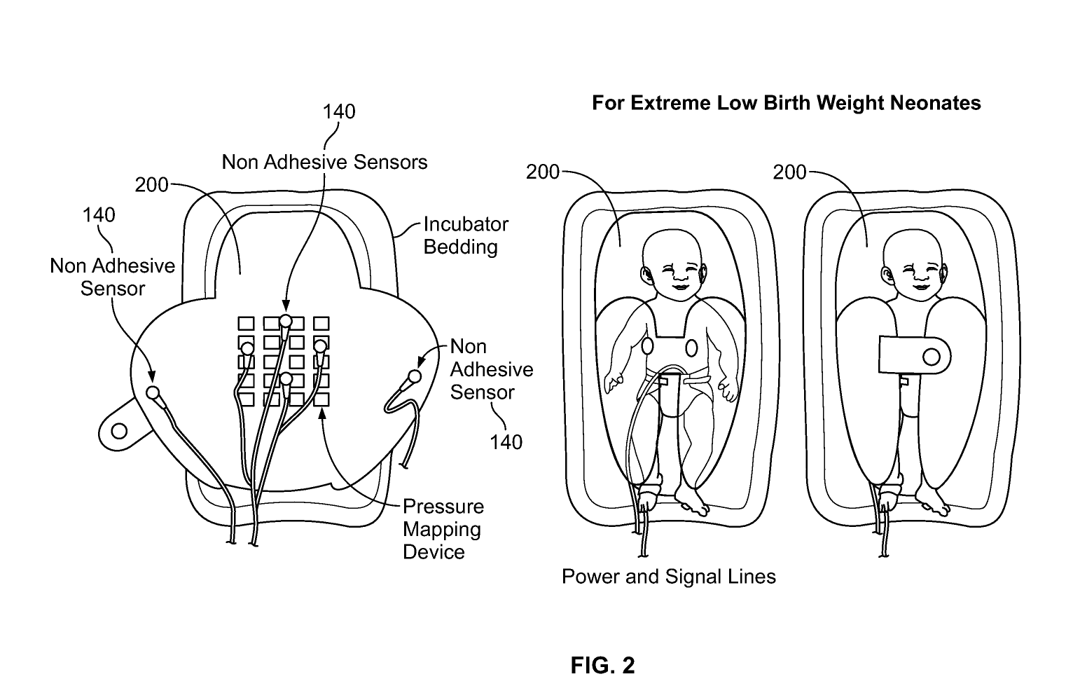

Referring to FIG. 2 and pursuant to the instant disclosure, there are two exemplary embodiments for incorporation of vibration sensors 140 associated with a patient. A first 200 of these two exemplary embodiments may be used with ELBW patients that are very small in size and extremely fragile. The substrate comprises bedding of the incubator (and optionally a cover as will be discussed in more detail hereafter) and is embedded with at least four vibration sensors 140 to monitor the heart and bowels as well as a pressure mapping device. In exemplary form, the pressure mapping device is fixed in position as part of the bedding, as are the vibration sensors. In this manner, the position of the pressure mapping device with respect to the vibration sensors is known. Accordingly, the pressure mapping device sends signals to the CADC 110 indicating the position of the infant. In this fashion, the CADC 110 receives signals as to the position of the infant and outputs of the vibration sensors 140 so that the CADC is operative to determined which of the vibration sensors (and its corresponding signal outputs) should be utilized to monitor what organs (e.g., heart, lungs, bowel, etc. ). For example, if the pressure mapping device senses that the infant is moved away from a particular location where a vibration sensor is positioned, the CADC will know to ignore or not poll that sensor for vibration signals. By way of example, the pressure mapping device may comprise an array of strain sensitive sensors, which may be based on capacitive (e.g., double plate capacitors , novel sensors), piezo-resistive (e.g., micro-cantilevers, micro-diaphragm, piezo-resistive ink) or electrical impedance tomography (e.g., electro-conductive knitted structure) technologies. In addition to the bedding underlying the infant, the bedding may include an infant cover are embedded with at least two additional vibration sensors 140 for monitoring the lungs. In this fashion, when the infant is laid on the bedding and wrapped in the cover, at least four vibration sensors 140 are monitoring the infant and sending signals to the CADC without necessitating the use of adhesives to attach the sensors to the infant in the incubator. As used herein, bedding generally encompasses the bedding the infant lies on top of in addition to covers placed over the infant.

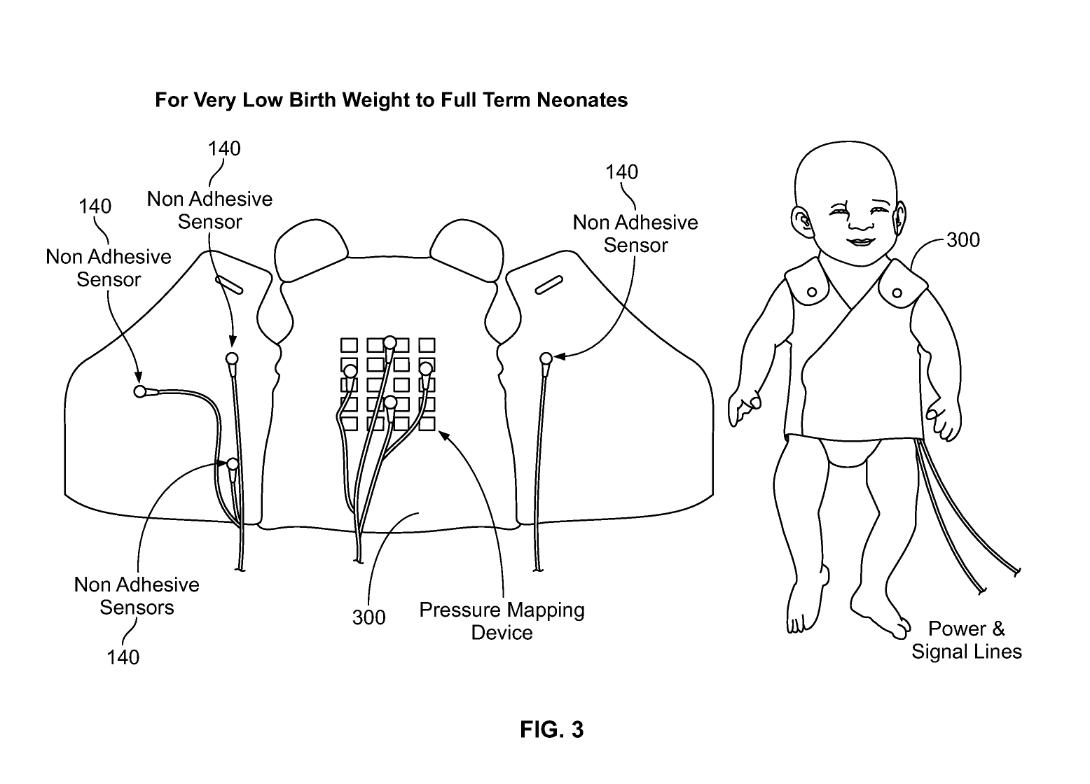

As shown in FIG. 3, a second exemplary embodiment 300 for embedding vibration sensors 140 comprises a flexible vest configured to be donned by low birth weight infants. In exemplary form, the fest includes a back section with shoulder straps and buttons, in addition to a pair of wrap-around sides with eyelets that are configured to overlap one another. More specifically, the eyelets are configured to receive an associated button of each shoulder strap to mount the back section to the wrap around sides. In this exemplary embodiment, the back section includes at least four vibration sensors 140 to monitor the heart and bowel as well as a pressure mapping device. In exemplary form, the pressure mapping device is fixed in position as part of the back section, as are the vibration sensors. In this manner, the position of the pressure mapping device with respect to the vibration sensors is known. Accordingly, the pressure mapping device sends signals to the CADC 110 indicating the position of the infant with respect to the flexible vest. In this fashion, the CADC 110 receives signals as to the position of the infant and outputs of the vibration sensors 140 so that the CADC is operative to determined which of the vibration sensors (and its corresponding signal outputs) should be utilized to monitor what organs (e.g., heart, lungs, bowel, etc.). For example, if the pressure mapping device senses that the infant is moved away from a particular location where a vibration sensor is positioned, the CADC will know to ignore or not poll that sensor for vibration signals. By way of example, the pressure mapping device may comprise an array of strain sensitive sensors, which may be based on capacitive (e.g., double plate capacitors, novel sensors), piezo-resistive (e.g., micro-cantilevers, micro-diaphragm, piezo-resistive ink) or electrical impedance tomography *e.g., electro-conductive knitted structure) technologies. In addition, the left wrap-around side includes a lung vibration sensor 140, a heart vibration sensor 140, and a bowel vibration sensor 140, while the right side wrap-around includes another lung vibration sensor 140. In this fashion, the left side wrap-around is positioned adjacent the torso of the infant first, followed by overlapping the right side wrap-around. In order to secure the wrap-arounds to one another. Velcro may be applied to the outside (opposite the side with the vibration sensors 140) of the left side wrap-around and to the inside (same side with the vibration sensor 140) of the right side wrap-around). Accordingly, outputs from the vibration sensors 140 and pressure mapping device are directed to the CADC 110.

Both exemplary embodiments 200, 300 allow the infant to move freely without restriction. However, as the parameters for diagnosis and classification vary with the targeting organs, it is important to identify the sensors with the monitoring organ. To achieve that, the substrate and flexible components may contain a pressure mapping device such as an isolated layer of conductive fabric. The pressure map may be used to monitor the general movement of the infant, determine the location of the closest sensors to the infant's heart, lungs, and bowel, and subsequently activate the sensors for monitoring.

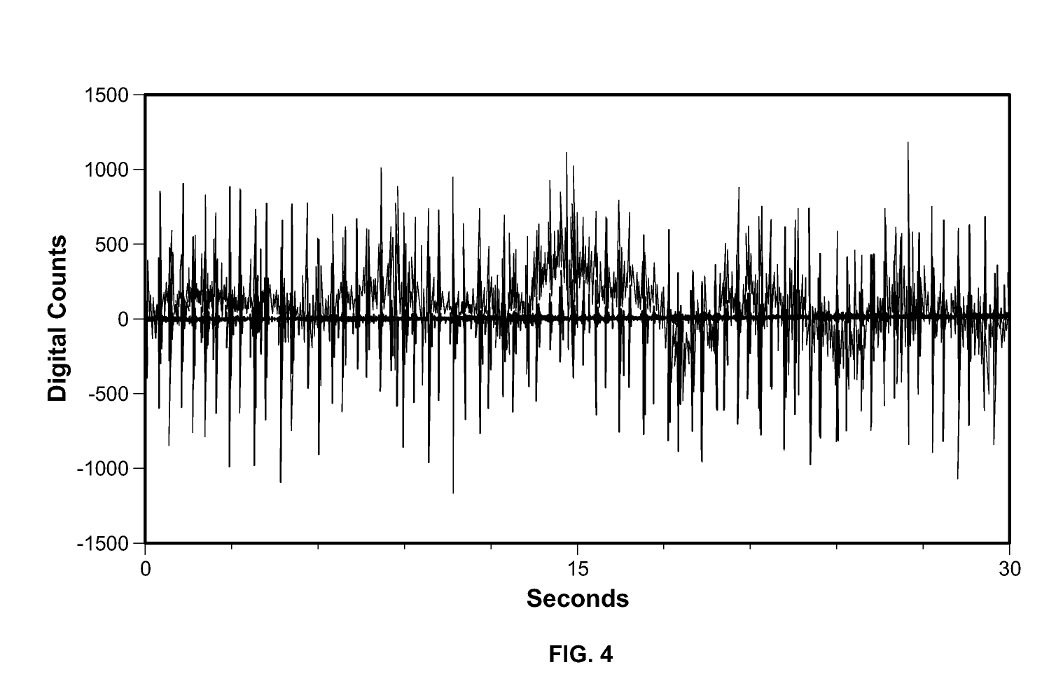

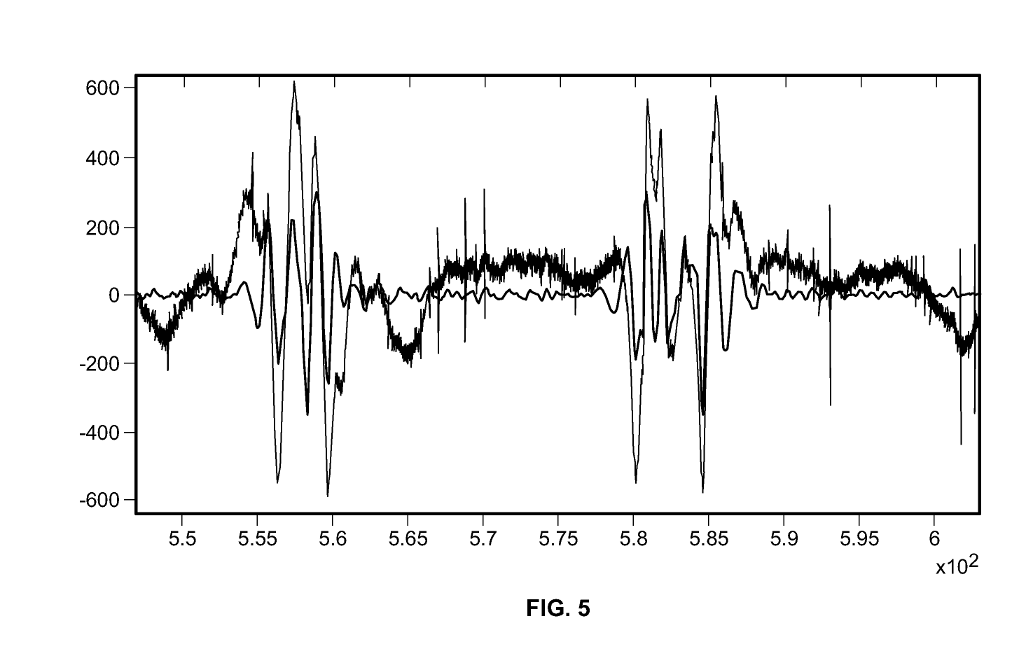

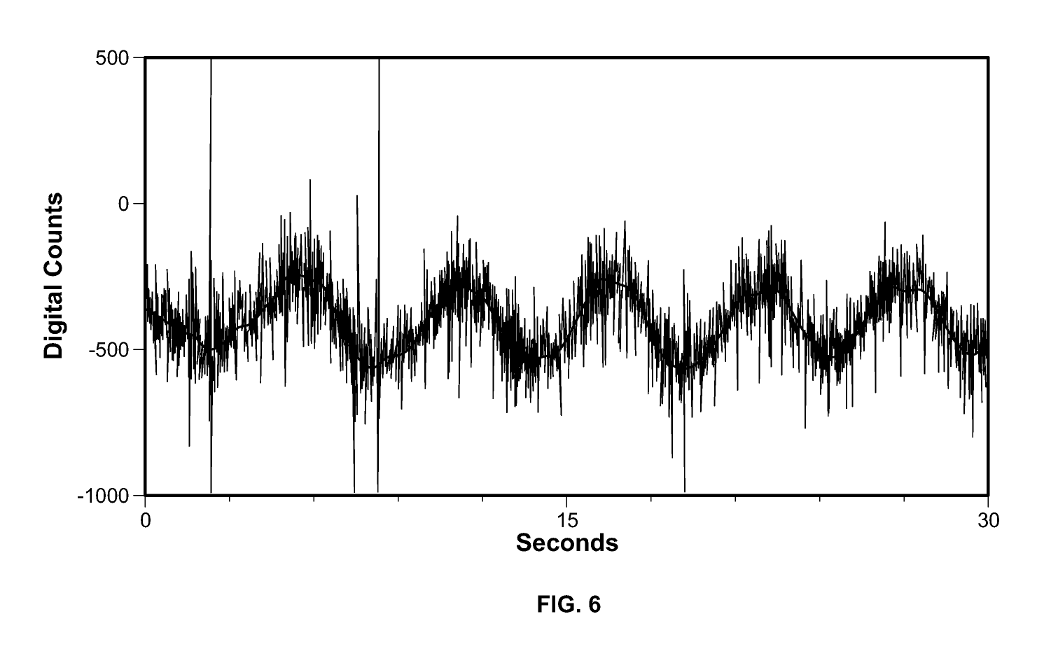

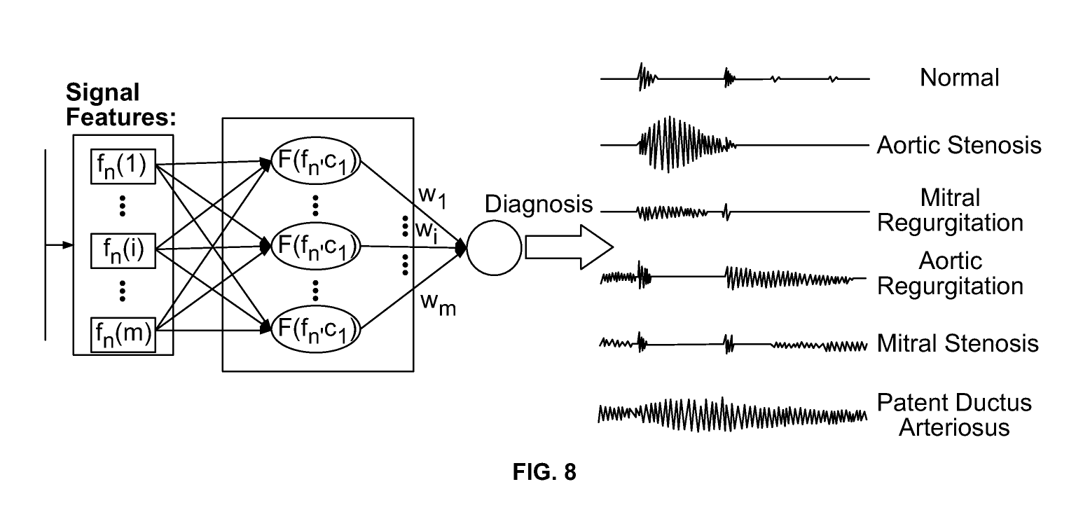

Referencing FIGS. 4-6, raw and filtered vibration signals obtained from a healthy human adult are depicted. The digitized vibration signals from the vibration sensors 140. This algorithm is operative to condition the signals from the vibration sensors and filter noise accompanying the vibration output data and filter the vibration output data based on the primary monitoring target (i.e., the heart, lungs, bowel, etc.). A second algorithm comprises an envelope extraction algorithm is applied to vibration sensors used to output data/signal concerning the patient's heart and lung functions. In particular, this algorithm determines the envelope of the processes vibration signal based upon characteristics of the incoming sensor data. A third algorithm comprises a segmentation algorithm that also applies to vibration sensors used to output data/signal concerning the patient's heart and lung functions. For heart and lungs monitoring applications, as the signals are periodic, an extraction algorithm for vibration segmentation is also applied to the filtered signals to determine physiological parameters of the signals. In exemplary form, the vibration data/signal is segmented for sound and classified using the enveloped signal. The segmented sound signal is used to determine heart rate and breathing rate, in addition to being an input for use with the diagnostic algorithm (the fifth algorithm). A fourth algorithm comprises the signal analysis algorithm that is applied to all vibration sensors. The signal analysis algorithm may be applied to raw, processed, enveloped, or segmented signals and is utilized to determine specific signal characteristics such as frequency components of the signal, amplitude levels, duration of the signal, frequency of the occurrences, and timing analysis. A fifth algorithm comprises a diagnostic algorithm that is operative to classify the signals using the segmented signals and the signal analysis algorithm output in order to determine a patient diagnosis. In addition to calculating vitals such as heart and breathing rates, the amplitude and the ratio of the systolic and diastolic durations may be used as inputs to a classification algorithm, where heart conditions such as aortic stenosis, mitral regurgitation, aortic regurgitation, mitral stenosis, and patent duetus arteriosus can be diagnosed such as those identified in FIG. 8. But the diagnostic algorithm may also be applied to other vibration sensors, such as the bowel vibration sensor, to diagnose conditions resulting from the absence of a bowel sound or too frequent of a bowel sound.

A significant advantage of using the exemplary patient monitoring system 100 is that data may be stored in a storage unit such as personal computer or server, and provides an excellent record of the patient's history. If an anomaly is detected, the processed signals may be digitally resampled to audible range and played back to the clinician or physician remotely without opening the incubator.

When an abnormal vital is detected, an event log is created and the information of the CADC is logged. The system 100 then alerts the caregiving staff that an anomaly has been detected, along with providing the preliminary diagnosis from the CADC.

For apnea prevention, a physical stimulation device 130 such as vibrating motor is embedded into the garment of the patient or otherwise placed in physical contact with the patient, which is triggered to restore breathing when an apnea event is determined by the CADC. The central issue that this system 100 addresses is the delay in care that is provided to the infant in the event of an apneic episode or other episode where time is of the essence. In the case of apnea using present day detection equipment, the delivery of care can take anywhere from 5-20 seconds after a breathing rate alarm sounds, or even longer depending on the circumstances of the caregiving staff. Every second lost is detrimental to the infant's health, due to the effects of hypoxemia and bradycardia. This system 100 completely eliminates this delay in care. Through real time alarm data monitoring, the exemplary system 100 can immediately detect an apneic episode and immediately trigger the stimulation device 130, thereby reinitiating normal breathing. The system 100 also alerts the caregiver of the episode, corrective action taken, and continues to monitor the vitals of the patient to determine if apnea has continued. In any event, the associated electronic sensors of the system 100 within the incubator are hermetically sealed to protect from the humid environment.

As discussed previously, the outputs of an existing vitals monitoring system 180 that may include an ECG, pulse oximeter 160, capnography 150, and thermal infrared camera 190 may be used as inputs to the CADC 110 as additional peripherals to aid the diagnostic classification algorithm. In the case of the thermal infrared camera 190, this device is used to take thermal images of the patient periodically in order to construct a heat map enabling non-instructive detection of certain vascular diseases.

Following from the above description and invention summaries, it should be apparent to those of ordinary skill in the art that, while the methods and apparatuses herein described constitute exemplary embodiments of the present invention, the invention contained herein is not limited to this precise embodiment and that changes may be made to such embodiments without departing from the scope of the invention as defined by the claims. Additionally, it is to be understood that the invention is defined by the claims and it is not intended that any limitations or elements describing the exemplary embodiments set forth herein are to be incorporated into the interpretation of any claim element unless such limitation or element is explicitly stated. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein.

What is claimed is:

1. A neonatal monitoring system comprising:
a substrate comprising at least one of bedding and a garment for a patient, the substrate including at least four vibration sensors and a pressure sensor array;
a computer communicatively coupled to the at least four vibration sensors to receive output data from each of the at least four vibration sensors, where the computer includes at least one algorithm for filtering and conditioning output data received from the at least four vibration sensors; and,
a visual display communicatively coupled to the computer for displaying information regarding the patient condition.

2. The neonatal monitoring system of claim 1, wherein the computer includes at least one of hardware and software for conditioning the output data, segmenting the output data, and analyzing the output data.

3. The neonatal monitoring system of claim 2, wherein the at least one of hardware and software generates processed data that is displayed by the visual display.

4. The neonatal monitoring system of claim 1, wherein the computer includes memory for recording historical patient conditions.

5. The neonatal monitoring system of claim 1, wherein the computer is communicatively coupled to a memory for recording historical patient conditions.

6. The neonatal monitoring system of claim 1, wherein the substrate comprises bedding including at least two of the at least four vibration sensors.

7. The neonatal monitoring system of claim 6, wherein the substrate also comprises the garment including at least two of the at least four vibration sensors.

8. The neonatal monitoring system of claim 1, wherein the substrate comprises bedding including the at least four vibration sensors.

9. The neonatal monitoring system of claim 1, wherein the computer utilizes the output data to generate audio data that representative of sounds indicative of the patient condition.

10. The neonatal monitoring system of claim 1, wherein the computer includes an algorithm to poll the at least four vibration sensors and allowing isolation of at least one vibration sensor of the at least four vibration sensors.

11. The neonatal monitoring system of claim 1, wherein the computer includes an algorithm generating an alert signal relayed to a caregiver interface to alert a caregiver of an undesirable patient condition.

12. The neonatal monitoring system of claim 11, wherein the caregiver interface comprises at least one of a visual display, a speaker, and a warning light.

13. The neonatal monitoring system of claim 1, further comprising a stimulation device communicatively coupled to the computer and selectively activated by the computer in response to the computer diagnosing an abnormal patient condition.

14. The neonatal monitoring system of claim 13, wherein the stimulation device comprises a vibrator.

15. The neonatal monitoring system of claim 1, wherein the computer include a diagnostic algorithm to diagnose a patient condition responsive to reception of the output data.

16. The neonatal monitoring system of claim 1, wherein the computer include a segmentation algorithm to segment the output data to isolate data into heart data and lung data and classify the heart data and the lung data.

17. The neonatal monitoring system of claim 1, wherein:
the substrate comprises bedding;
the bedding includes a cover and an underlying constituent;
the underlying constituent includes the pressure sensor array and at least four vibration sensors; and,
the cover includes at least two vibration sensors.

18. A method of monitoring and curtailing apnea in a patient comprising:
monitoring breathing of a patient using at least one vibration sensor;
utilizing a signal from the at least one vibration sensor to diagnose apnea;
responsive to diagnosing apnea, automatically powering a stimulation device in contact with the patient to restore breathing;
verifying restoration of breathing by again monitoring breathing of the patient using the at least one vibration sensor.

19-20. (canceled)

21. A method of monitoring a neonatal patient comprising:
monitoring a lung of a neonatal patient using a first vibration sensor;
monitoring a heart the neonatal patient using a second vibration sensor;
monitoring a bowel of the neonatal patient using a third vibration sensor;
utilizing a signals from the first, second, and third vibration sensors to reflect a condition of at least one of the heart, lung, and bowel of the neonatal patient; and,
displaying data representative of the condition of the neonatal patient.
